(19) United States
(12) Patent Application Publication
(10) Pub. No.: US 2024/0159412 A1
(43) Pub. Date: May 16, 2024

(54) SYSTEM AND METHOD FOR DETECTING CONTAMINATED DUCTS WITHIN AN INDOOR ENVIRONMENT

(71) Applicant: Carrier Corporation, Palm Beach Gardens, FL (US)

(72) Inventors: Kanna Selvakani, Theni (IN); Rakesh Annavaram, Hyderabad (IN); Rakesh Kumar Arepalli, Hyderabad (IN); Jagadish Reddy Putta, Hyderabad (IN)

(21) Appl. No.: 18/510,398

(22) Filed: Nov. 15, 2023

Related U.S. Application Data
(60) Provisional application No. 63/383,848, filed on Nov. 15, 2022.

(51) Int. Cl. F24F 11/52; F24F 11/30; F24F 11/72

(52) U.S. Cl. CPC F24F 11/52; F24F 11/30; F24F 11/72; F24F 2110/50

(57) ABSTRACT

An HVAC system that includes an air handling unit (AHU), indoor air quality (IAQ) sensors installed in one or more zones within an indoor environment, and a control device. The control device controls the IAQ sensors to measure a first set of IAQ values when air circulation to the one or more zones is turned off and controls the IAQ sensors to measure a second set of IAQ values when the air circulation to the one or more zones is turned on. The control device further compares the first set of measured IAQ values with the second set of measured IAQ values and detects at least one contaminated duct based on a result of the comparison. The control device further notifies, on a display screen or on a GUI of an application using a communication interface, a message indicating the at least one contaminated duct to a user.

### FIELD OF THE INVENTION

The disclosure relates to the field of Heating, ventilation, and air conditioning (HVAC) systems. In particular, the disclosure relates to a system and method for detecting contaminated ducts within an indoor environment.

### BACKGROUND

A Duct system is used for circulating conditioned air between HVAC systems and indoor environments such as residential, industrial, or office rooms. Dirty air ducts hinder the HVAC system and user's comfort over a certain period of time due to dust, germs, and contaminants that clog the duct channels.

It is challenging to detect dirty air duct issues, especially in an indoor environment having a large installation area and requires a lot of manual investigation. It would be advantageous to provide a method and system that may detect contaminated air ducts within the indoor environment.

### SUMMARY

This summary is provided to introduce a selection of concepts in a simplified format that are further described in the detailed description of the invention. This summary is not intended to identify key or essential inventive concepts of the invention, nor is it intended for determining the scope of the invention.

Disclosed is an HVAC system for detecting at least one contaminated duct among one or more ducts within an indoor environment. The HVAC system includes an air handling unit (AHU), a plurality of indoor air quality (IAQ) sensors installed in one or more zones, and a control device that includes a processor, a communication interface, and a display screen. The processor is configured to control the AHU to turn-off air circulation to one or more zones within the indoor environment for a first time period and control the plurality of IAQ sensors to measure a first set of IAQ values in the one or more zones when the air circulation to the one or more zones is turned off. The processor is further configured to control the AHU to turn-on the air circulation to the one or more zones for a second time period and control the plurality of IAQ sensors to measure a second set of IAQ values in the one or more zones when the air circulation to the one or more zones is turned on. The processor is further configured to compare the first set of measured IAQ values with the second set of measured IAQ values and then detect the at least one contaminated duct among the one or more ducts based on a result of the comparison between the first set of measured IAQ values and the second set of measured IAQ values. Thereafter, the processor is further configured to notify, based on the detection of the at least one contaminated duct, a message indicating the at least one contaminated duct to a user on the display screen or a GUI of an application using the communication interface.

In one or more embodiments, the processor may be further configured to detect a specific location of the at least one contaminated duct within the indoor environment, and then notify, via the GUI of the application using the communication interface or on the display screen, a message indicating the detected location of the at least one contaminated duct to the user.

In one or more embodiments, for detecting the specific location of the at least one contaminated duct, the processor may be further configured to re-control the AHU to turn-off the air circulation to corresponding zones among the one or more zones for a third time period, and control each of the plurality of IAQ sensors to measure a first set of individual IAQ values of the corresponding zones when the air circulation to the corresponding zones is turned off for the third time period. Thereafter, the processor may be further configured to re-control the AHU to turn-on the air circulation to the corresponding zones for a fourth time period, and control each of the plurality of IAQ sensors to measure a second set of individual IAQ values of the corresponding zones when the air circulation to the corresponding zones is turned-on for the fourth time period. Once the first set of individual IAQ values and the second set of individual IAQ values are measured, the processor may be further configured to compare the first set of individual IAQ values with the second set of individual IAQ values and then detect the specific location of the at least one contaminated duct within the indoor environment based on a result of the comparison between the first set of individual IAQ values and the second set of individual IAQ values.

In one or more embodiments, the processor may be further configured to determine, based on the comparison between the first set of individual IAQ values and the second set of individual IAQ values, at least one IAQ value among the second set of individual IAQ values that is greater than at least one IAQ value among the first set of individual IAQ values. Thereafter, the processor may be further configured to detect the specific location of the at least one contaminated duct based on the at least one IAQ value that is determined among the second set of individual IAQ values.

In one or more embodiments, the processor may be further configured to periodically send each of the first set of measured IAQ values, the second set of measured IAQ values, the first set of individual IAQ values, and the second set of individual IAQ values to a cloud server using the communication interface.

In one or more embodiments, the plurality of sensors includes at least one of carbon dioxide sensor, PM2.5 sensor, PM10 sensor, or a total volatile organic compound (TVOC) sensor.

In one or more embodiments, the processor may be further configured to determine a ramping pattern of the second set of measured IAQ values with respect to a predefined required IAQ level and then detect one of a zone specific issue or an issue related to the at least one contaminated duct based on the determined ramping pattern.

Also disclosed herein is a method for detecting at least one contaminated duct among one or more ducts within an indoor environment. The method includes controlling an air handling unit (AHU) of an HVAC system to turn-off air circulation to one or more zones within the indoor environment for a first time period, and controlling a plurality of indoor air quality (IAQ) sensors of the HVAC system to measure a first set of IAQ values in the one or more zones when the air circulation to the one or more zones is turned off. The method further includes controlling the AHU to turn-on the air circulation to the one or more zones for a second time period, and controlling the plurality of IAQ sensors to measure a second set of IAQ values in the one or more zones when the air circulation to the one or more zones is turned on. The method further includes comparing the first set of measured IAQ values with the second set of measured IAQ values and thereafter detecting the at least one contaminated duct among the one or more ducts based on a result of the comparison between the first set of measured IAQ values and the second set of measured IAQ values. Once the at least one contaminated duct is detected, the method further includes notifying, based on the detection of the at least one contaminated duct, a message indicating the at least one contaminated duct to a user on the display screen or a GUI of an application using the communication interface.

In one or more embodiments, the method may further include detecting a specific location of the at least one contaminated duct within the indoor environment and thereafter notifying, on the GUI of the application using the communication interface or on the display screen, a message indicating the detected location of the at least one contaminated duct to the user.

In one or more embodiments, for detecting the specific location of the at least one contaminated duct, the method may further include re-controlling the AHU to turn-off the air circulation to corresponding zones among the one or more zones for a third time period, controlling each of the plurality of IAQ sensors to measure a first set of individual IAQ values of the corresponding zones when the air circulation to the corresponding zones is turned-off for the third time period. Thereafter, the method may further include re-controlling the AHU to turn-on the air circulation to the corresponding zones for a fourth time period, and controlling each of the plurality of IAQ sensors to measure a second set of individual IAQ values of the corresponding zones when the air circulation to the corresponding zones is turned-on for the fourth time period. Once the first set of individual IAQ values and the second set of individual IAQ values are measured, the method may further include comparing the first set of individual IAQ values with the second set of individual IAQ values and thereafter detecting the specific location of the at least one contaminated duct within the indoor environment based on a result of the comparison between the first set of individual IAQ values and the second set of individual IAQ values.

In one or more embodiments, the method may further include determining, based on the comparison between the first set of individual IAQ values and the second set of individual IAQ values, at least one IAQ value among the second set of individual IAQ values that is greater than at least one IAQ value among the first set of individual IAQ values, and thereafter detecting the specific location of the at least one contaminated duct based on the at least one IAQ value that is determined among the second set of individual IAQ values.

In one or more embodiments, the method may further include periodically sending each of the first set of measured IAQ values, the second set of measured IAQ values, the first set of individual IAQ values, and the second set of individual IAQ values to a cloud server using the communication interface.

In one or more embodiments, the plurality of sensors includes at least one of a carbon dioxide sensor, PM2.5 sensor, PM10 sensor, or a total volatile organic compound (TVOC) sensor.

In one or more embodiments, the method may further include determining a ramping pattern of the second set of measured IAQ values with respect to a predefined required IAQ level, and thereafter detecting one of a zone specific issue or an issue related to the at least one contaminated duct based on the determined ramping pattern.

To further clarify the advantages and features of the method and system, a more particular description of the method and system will be rendered by reference to specific embodiments thereof, which are illustrated in the appended drawing. It is appreciated that these drawings depict only typical embodiments of the invention and are therefore not to be considered limiting its scope. The invention will be described and explained with additional specificity and detail with the accompanying drawings.

### BRIEF DESCRIPTION OF THE DRAWINGS

These and other features, aspects, and advantages will become better understood when the following detailed description is read with reference to the accompanying drawings in which like characters represent like parts throughout the drawings, wherein:

FIG. 1 illustrates an example block diagram of a control system for air handling in the HVAC;

FIG. 2 illustrates another example block diagram of a control system in the HVAC;

FIG. 3 illustrates a block diagram of the AHU;

FIG. 4 illustrates a flowchart of method steps for detecting at least one contaminated duct among one or more ducts within an indoor environment;

FIG. 5 illustrates an example graphical representation of a ramping pattern when the air circulation is on;

FIG. 6 illustrates another example graphical representation of the ramping pattern when the air circulation is on and off, respectively; and

FIG. 7 illustrates a flowchart of method steps for detecting contaminated duct issues or zone specific issues using the ramping pattern.

Further, skilled artisans will appreciate that elements in the drawings are illustrated for simplicity and may not have necessarily been drawn to scale. For example, the flow charts illustrate the method in terms of the most prominent steps involved to help to improve understanding of aspects of the invention. Furthermore, in terms of the construction of the device, one or more components of the device may have been represented in the drawings by conventional symbols, and the drawings may show only those specific details that are pertinent to understanding the embodiments of the invention so as not to obscure the drawings with details that will be readily apparent to those of ordinary skill in the art having the benefit of the description herein.

### DETAILED DESCRIPTION

It should be understood at the outset that although illustrative implementations of embodiments are illustrated below, the system and method may be implemented using any number of techniques. The disclosure should in no way be limited to the illustrative implementations, drawings, and techniques illustrated below, including the exemplary design and implementation illustrated and described herein, but may be modified within the scope of the appended claims along with their full scope of equivalents.

The term “some” and “one or more” as used herein is defined as “one, or more than one, or all.” Accordingly, the terms “one,” “more than one,” but not all” or “all” would all fall under the definition of “some.” The term “some embodiments” or “one or more embodiments” may refer to one embodiment or several embodiments or all embodiments. Accordingly, the term “some embodiments” is defined as meaning “one embodiment, or more than one embodiment, or all embodiments.”

The terminology and structure employed herein are for describing, teaching, and illuminating some embodiments and their specific features and elements and do not limit, restrict, or reduce the spirit and scope of the claims or their equivalents.

More specifically, any terms used herein such as but not limited to “includes,” “comprises,” “has,” “have” and other grammatical variants thereof do not specify an exact limitation or restriction and certainly do not exclude the possible addition of one or more features or elements, unless otherwise stated, and must not be taken to exclude the possible removal of one or more of the listed features and elements, unless otherwise stated with the limiting language “must comprise” or “needs to include.”

The term “unit” used herein may imply a unit including, for example, one of hardware, software, and firmware or a combination of two or more of them. The “unit” may be interchangeably used with a term such as logic, a logical block, a component, a circuit, and the like. The “unit” may be a minimum system component for performing one or more functions or may be a part thereof.

Unless otherwise defined, all terms, and especially any technical and/or scientific terms, used herein may be taken to have the same meaning as commonly understood by one having ordinary skill in the art.

Embodiments will be described below in detail with reference to the accompanying drawings.

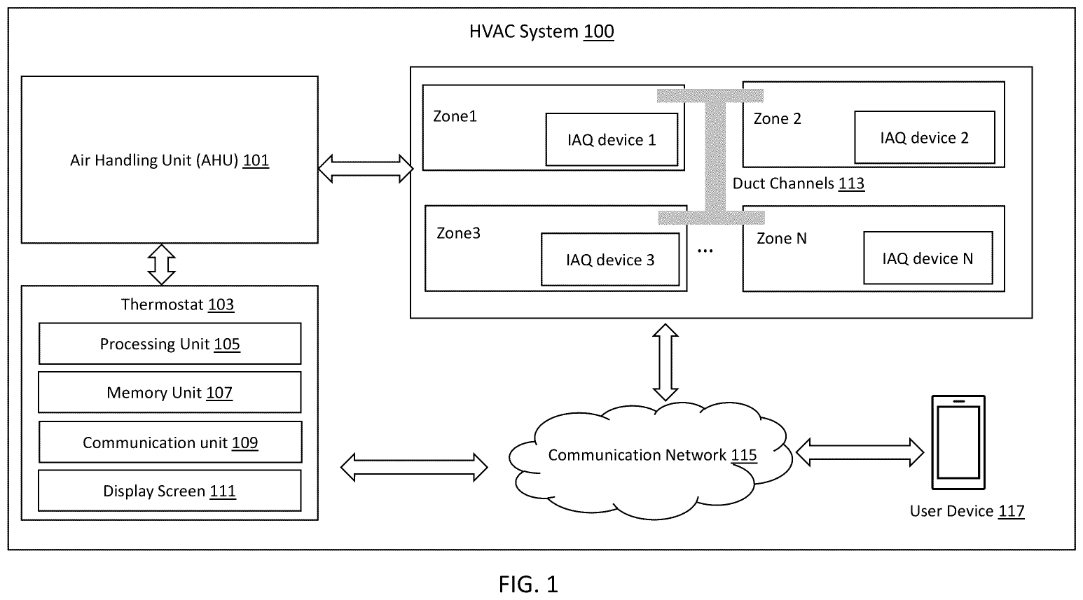

FIG. 1 illustrates an example block diagram of a control system for air handling in the HVAC. FIG. 1 depicts an HVAC system 100 that includes an AHU 101, a thermostat 103, a communication network 115, duct channels 113, and a user device 117. The Thermostat 103 includes a processing unit 105, a memory unit 107, a communication unit 109, a display screen 111, and a plurality of zones (Zone 1, Zone 2, Zone 3, . . . , Zone N) each including an IAQ device (IAQ device 1, IAQ device 2, IAQ device 3, . . . , IAQ device N), respectively. Each of the corresponding IAQ devices is installed within respective zones among the plurality of zones. Each of the corresponding IAQ devices includes an IAQ sensor.

As a non-limiting example, the plurality of zones corresponds to one or more zones within indoor environments such as residential buildings, industrial buildings, office rooms, corporate buildings, and the like. The duct channels 113 corresponds to one or more duct channels that carry cooled air from the AHU 101 throughout the zones within the indoor environment and are responsible for removing warm or stale air from the indoor environment to the outside.

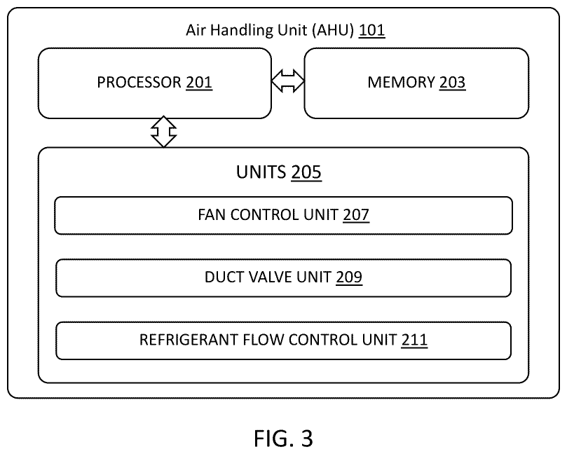

The AHU 101 corresponds to any now known or later developed air-handling unit for residential, industrial, or office use. The AHU 101 includes return air input, fresh air input, air mixing section, filters, cooling coils, heating coils, dampers or actuators, attenuator, discharge, one or more fan controlling units, one or more duct valve units, and one or more refrigerant flow control units. Additional, different, or fewer components may be provided. An example block diagram of the AHU 101 is shown in FIG. 3 of the drawings.

The thermostat 103 is configured to control the HVAC system 100 through a number of control circuits. The processing unit 105 of the thermostat 103 is adapted and programmed to control the HVAC system 100 and to carry out the techniques described in detail herein. The processing unit 105 may correspond to a processor. The processor can be a single processing unit or several units, all of which could include multiple computing units. Accordingly, the processing unit 105 may be implemented as one or more microprocessors, microcomputers, microcontrollers, digital signal processors, central processing units, state machines, logic circuitries, and/or any devices that manipulate signals based on operational instructions. Among other capabilities, the processing unit is configured to fetch and execute computer-readable instructions and data stored in the memory unit 107.

The communication network 115 may comprise a single, local network, a large network, or a plurality of small or large networks interconnected together. The communication network 115 may also comprise any type or number of local area networks (LANs) broadband networks, wide area networks (WANs), etc. Further, the communication network 115 may incorporate one or more LANs, and wireless portions and may incorporate one or more of various protocols and architectures such as TCP/IP, Ethernet, etc. The communication network 115 may also incorporate other types of public networks, such as the public switch telephone network (PSTN) or the like. The communication network 115 may further include a network interface to communicate via offline and online wireless communication with networks, such as the Internet, an Intranet, and/or a wireless network, such as a cellular telephone network, a wireless local area network (WLAN), personal area network, and/or a metropolitan area network (MAN). The wireless communication may use any of a plurality of communication standards, protocols, and technologies, such as Global System for Mobile Communications (GSM), Enhanced Data GSM Environment (EDGE), wideband code division multiple access (W-CDMA), code division multiple access (CDMA), LTE, time division multiple access (TDMA), Bluetooth, Wireless Fidelity (Wi-Fi) (such as IEEE 802.11, IEEE 802.11b, IEEE 802.11g, IEEE 802.11n, and/or any other IEEE 802.11 protocol), voice over Internet Protocol (VoIP), Wi-MAX, Internet-of-Things (IoT) technology, Machine-Type-Communication (MTC) technology, a protocol for email, instant messaging, and/or Short Message Service (SMS).

The user device 117 may be wirelessly connected to the HVAC system 100 via the communication network 115 and may correspond to at least one portable electronic device or non-portable electronic device used by the user to send a control command to the HVAC system 100 or may receive an update from the HVAC system 100.

The memory unit 107 corresponds to a memory that includes one or more computer-readable storage media. The memory may include non-volatile storage elements. Examples of such non-volatile storage elements may include magnetic hard discs, optical discs, floppy discs, flash memories, or forms of electrically programmable memories (EPROM) or electrically erasable and programmable (EEPROM) memories. In addition, the memory may, in some examples, be considered a non-transitory storage medium. The term “non-transitory” may indicate that the storage medium is not embodied in a carrier wave or a propagated signal. However, the term “non-transitory” should not be interpreted to mean that the memory is non-movable. In certain examples, a non-transitory storage medium may store data that can, over time, change (e.g., in Random Access Memory (RAM) or cache.

The communication unit 109 is configured to communicate voice, video, audio, images, or any other data over the communication network 115. Further, the communication unit 109 may include a communication port or a communication interface for sending and receiving notifications to the user device 117 via the communication network 115. The communication port or the communication interface may be a part of the processing unit 105 or maybe a separate component. The communication port may be created in software or maybe a physical connection in hardware. The communication port may be configured to connect with the communication network 115, external media, the display screen 111, or any other components in the HVAC system 100, or combinations thereof. The connection with the communication network 115 may be a physical connection, such as a wired Ethernet connection or may be established wirelessly as discussed above. Likewise, the additional connections with other components of the HVAC system 100 may be physical or may be established wirelessly.

The display screen 111 is configured to display an image or information associated with the operations of the thermostat 103. As a non-limiting example, the display screen 111 may be Light Emitting Diode (LED), Liquid Crystal Display (LCD), Organic Light Emitting Diode (OLED), Active Matrix Organic Light Emitting Diode (AMOLED), or Super Active Matrix Organic Light Emitting Diode (SAMOLED) screen. The display screen 111 may be of varied resolutions.

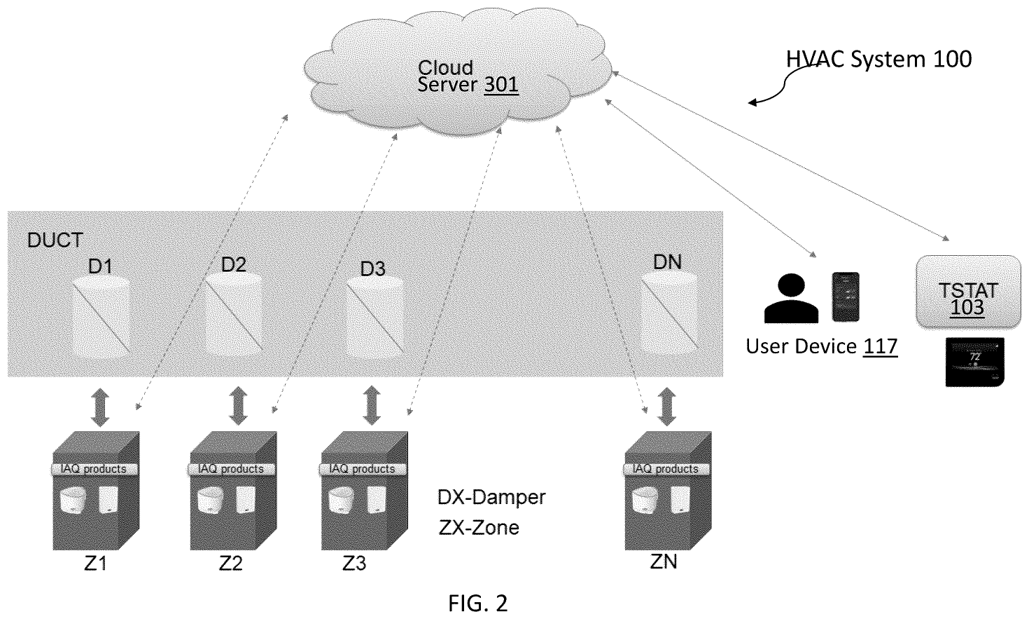

FIG. 2 illustrates another example block diagram of a control system in the HVAC 100, in accordance with an embodiment of the disclosure. According to FIG. 2, the HVAC system 100 includes a cloud server 301, the thermostat 103, the user device, a plurality of zones (Z1, Z2, Z3, . . . , ZN), a plurality of ducts (D1, D2, D3, . . . , DN) in which one or more IAQ devices are installed. The functionalities and operations performed by the components and units of the HVAC system 100 of FIG. 2 other than the cloud server 301 are similar to that of the HVAC system 100 of FIG. 1. Therefore, a description of the functionalities and the operations performed by the components and units of the HVAC system other than the cloud server 301 is omitted herein for the sake of brevity and ease of explanation.

The cloud server 301 is a virtual server having a cloud database to store the IAQ values measured at different time intervals by corresponding IAQ devices installed in the corresponding ducts during the on and off state of the AHU 101. The thermostat 103 is configured to detect at least one contaminated duct among the ducts D1 to DN by comparing a first set of measured IAQ values and a second set of measured IAQ values. The first set of measured IAQ values includes IAQ values of the corresponding zones Z1 to ZN when the air circulation to the one or more zones Z1 to ZN is turned off by the AHU 101. The second set of measured IAQ values includes IAQ values of the corresponding zones Z1 to ZN when the air circulation to the one or more zones Z1 to ZN is turned on by the AHU 101.

Each of the IAQ devices is a standalone device or may be integrated into the thermostat 103. Multiple IAQ sensors are placed at various locations within the duct channels strategically to detect IAQ levels of different zones within the indoor environment. As a non-limiting example, the IAQ sensors comprise a carbon dioxide sensor (CO2) sensor, PM2.5/PM10 sensors, a total volatile organic compound (TVOC) sensor, etc., and are configured to periodically send all the measured IAQ values/levels to the cloud server 301 via the communication interfaces (such as the communication network 115).

FIG. 3 illustrates a detailed block diagram of the AHU 101, in accordance with one or more embodiments of the disclosure. The AHU 101 includes a processor 201, a memory 203, units 205, a fan control unit 207, a duct valve unit 209, and a refrigerant flow control unit 211.

The processor 201 can be a single processing unit or several units, all of which could include multiple computing units. The processor 201 may be implemented as one or more microprocessors, microcomputers, microcontrollers, digital signal processors, central processing units, state machines, logic circuitries, and/or any devices that manipulate signals based on operational instructions. Among other capabilities, the processor 201 is configured to fetch and execute computer-readable instructions and data stored in the memory 203.

The memory 203 may include any non-transitory computer-readable medium known in the art including, for example, volatile memory, such as static random-access memory (SRAM) and dynamic random-access memory (DRAM), and/or non-volatile memory, such as read-only memory (ROM), erasable programmable ROM, flash memories, hard disks, optical disks, and magnetic tapes.

The units 205, amongst other things, include routines, programs, objects, components, data structures, etc., which perform particular tasks or implement data types. The units 205 may also be implemented as, signal processor(s), state machine(s), logic circuitries, and/or any other device or component that manipulates signals based on operational instructions.

The units 205 can be implemented in hardware, instructions executed by a processing unit, or by a combination thereof. The processing unit can comprise a computer, a processor, such as the processor 201, a state machine, a logic array, or any other suitable devices capable of processing instructions. The processing unit can be a general-purpose processor which executes instructions to cause the general-purpose processor 201 to perform the required tasks or, the processing unit can be dedicated to performing the required functions.

The fan control unit 207 is configured to control the fan installed in the AHU 101 to change a flow rate of the air.

The duct valve unit 209 is configured to control valves in the duct channels 113 to supply or cut off the air to the one or more zones (Z1 through ZN).

The refrigerant flow control unit 211 is configured to control a flow rate of cold refrigerant or hot refrigerant in an evaporator tube of the AHU 101.

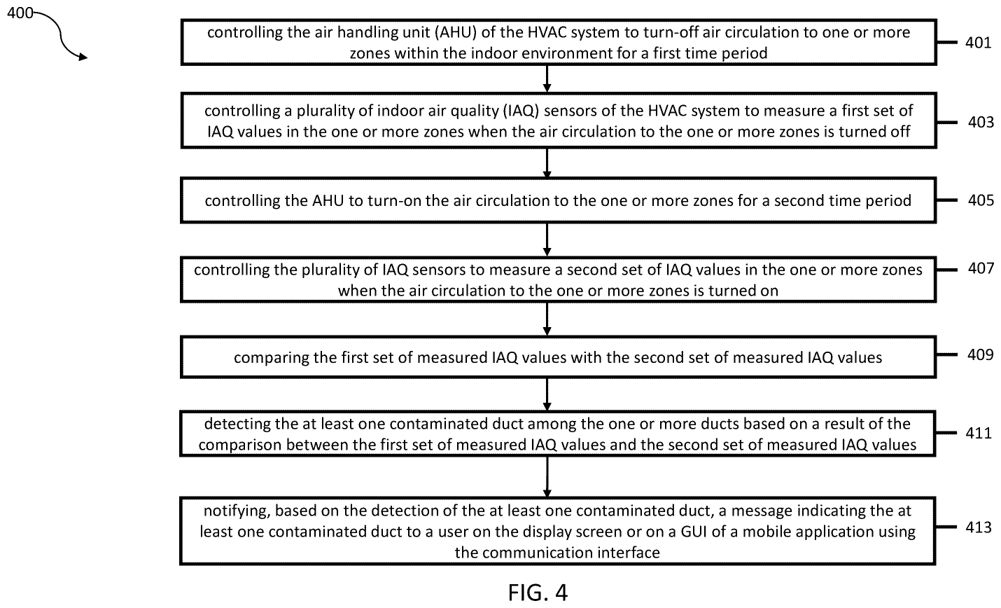

FIG. 4 illustrates a flowchart of method steps for detecting at least one contaminated duct among the one or more ducts within the indoor environment. FIG. 4 depicts a method 400 that is executed by the processing unit 105 of the thermostat 103 and the AHU 101 of FIG. 1 of the drawings.

At step 401 of method 400, the processing unit 105 controls the AHU 101 to turn-off the air circulation to the one or more zones within the indoor environment for a first time period. The flow of the method 300 now proceeds to (step 403).

At step 403, the processing unit 105 further controls the plurality of IAQ sensors to measure a first set of IAQ values in the one or more zones when the air circulation to the one or more zones is turned off by the AHU 101. The flow of the method 400 now proceeds to (step 405).

At step 405, after the measurement of the first set of IAQ values in the one or more zones, the processing unit 105 further controls the AHU 101 to turn-on the air circulation to the one or more zones for a second time period. The flow of the method 400 now proceeds to (step 407).

At step 407, the processing unit 105 further controls the plurality of IAQ sensors to measure a second set of IAQ values in the one or more zones when the air circulation to the one or more zones is turned on by the AHU 101. The flow of the method 400 now proceeds to (step 409).

At step 409, the processing unit 105 further compares the first set of measured IAQ values with the second set of measured IAQ values. The flow of the method 400 now proceeds to (step 411).

At step 411, the processing unit 105 detects the at least one contaminated duct among the one or more ducts (D1 to DN) based on a result of the comparison between the first set of measured IAQ values and the second set of measured IAQ values. The flow of the method 400 now proceeds to (step 413).

At step 413, once it is detected that there is a presence of at least one contaminated duct among the one or more ducts (D1 to DN), the processing unit 105 further notifies a message indicating the at least one contaminated duct to a user on the display screen 111 or on a GUI of an application installed in the user device 117 using the communication interface of the communication unit 109.

In particular, when air circulation is turned OFF by the AHU 101, IAQ values are measured by all the IAQ devices and sent to the thermostat 103, where an average of the IAQ value from all the zones (Z1 to ZN) is computed. This average measurement is used as a baseline to compare the IAQ values from the at least one contaminated duct. If the IAQ values in at least one of the zones are more than the computed average IAQ value when the air circulation is turned ON by the AHU 101, then the thermostat detects that the zone with the IAQ level greater than the average IAQ level is a zone having the contaminated duct. As a non-limiting example, assuming an average IAQ value is equal to 50 and the measured IAQ value from a particular zone is 150, then the particular zone will be considered as a zone having the contaminated duct. Those skilled in the art will appreciate that the aforementioned example is merely exemplary and is not intended to limit the scope of the invention.

According to some embodiments, the processing unit 105 may detect a specific location of the at least one contaminated duct within the indoor environment and notify a message indicating the detected location to the user on the GUI of the application using the communication interface or on the display screen 111.

According to some embodiments, the processing unit 105 may perform a series of control steps for detecting the specific location of the at least one contaminated duct within the indoor environment.

For detecting the specific location of the at least one contaminated duct, at first, the processing unit 105 re-controls the AHU 101 to turn-off the air circulation to corresponding zones among the one or more zones for a third time period. Secondly, the processing unit 105 re-control each of the plurality of IAQ sensors to measure a first set of individual IAQ values of the corresponding zones when the air circulation to the corresponding zones is turned off by the AHU 101 for the third time period. Thirdly, the processing unit 105 re-controls the AHU 101 to turn-on the air circulation to the corresponding zones for a fourth time period, and fourthly, re-controls each of the plurality of IAQ sensors to measure a second set of individual IAQ values of the corresponding zones when the air circulation to the corresponding zones is turned-on by the AHU 101 for the fourth time period. Fifthly, the processing unit 105 compares the first set of individual IAQ values with the second set of individual IAQ values, and finally detects the specific location of the at least one contaminated duct within the indoor environment based on a result of the comparison between the first set of individual IAQ values and the second set of individual IAQ values.

According to some embodiments, the processing unit 105 may determine, based on the comparison between the first set of individual IAQ values and the second set of individual IAQ values, at least one IAQ value among the second set of individual IAQ values that is greater than at least one IAQ value among the first set of individual IAQ values, and thereafter detects the specific location of the at least one contaminated duct based on the at least one IAQ value that is determined among the second set of individual IAQ values.

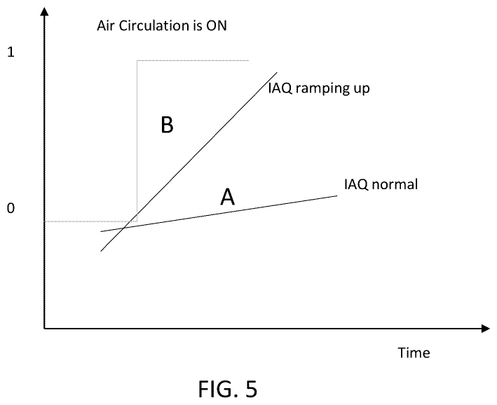

According to some embodiments, the processing unit 105 may also detect one of a zone specific issue or an issue related to the at least one contaminated duct using a ramping pattern determination process. In the detection of one of the zone specific issues or the issue related to the at least one contaminated duct, the processing unit 105 performs a series of steps described below herein in accordance with FIGS. 5 to 7 of the drawings.

FIG. 5 illustrates an example graphical representation of a ramping pattern when the air circulation is on, respectively. As can be seen in FIG. 5, two cases A and B are illustrated. In case A and case B, the processing unit 105 controls the AHU 101 to turn on the air circulation to the one or more zones and checks for the IAQ PM2.5 pattern. If it is determined by the processing unit 105 that the IAQ PM2.5 pattern is not ramping up and may go down, the processing unit 101 considers it as indented. Further, if it is determined by the processing unit 105 that the IAQ PM2.5 pattern is ramping up, the processing unit 101 considers it as a duct issue.

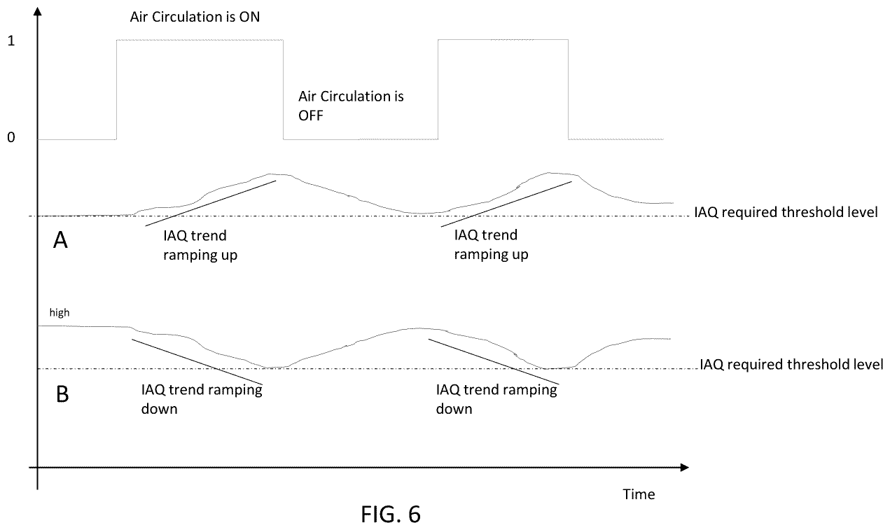

Similarly, FIG. 6 illustrates another example graphical representation of the ramping pattern when the air circulation is on and off, respectively. As can be seen in FIG. 6, two cases A and B are illustrated. In each case A and case B of FIG. 6, the processing unit 105 periodically controls the AHU 101 to turn on the air circulation to the one or more zones and checks for the IAQ PM2.5 pattern. If it is determined by the processing unit 105 that the IAQ PM2.5 pattern is ramping up in comparison to a predefined required IAQ threshold level, the processing unit 105 considers it as the duct issue. Further, if it is determined by the processing unit 105 that the IAQ PM2.5 pattern is ramping down from a higher side of the predefined required IAQ threshold level, the processing unit 105 considers it as the zone specific issue. The predefined required threshold IAQ levels correspond to an average IAQ value of all the zones within the indoor environment. The processing unit 105 uses this threshold for determining an increase or a decrease in the ramping pattern to detect whether the issue is a zone specific issue or an issue related to the at least one contaminated duct.

As a non-limiting example, the zone specific issues may include, but are not limited to, an open window/door within the indoor environment, a type of paint used on walls of the indoor environment, bad odors related to substances within the indoor environment, and the like.

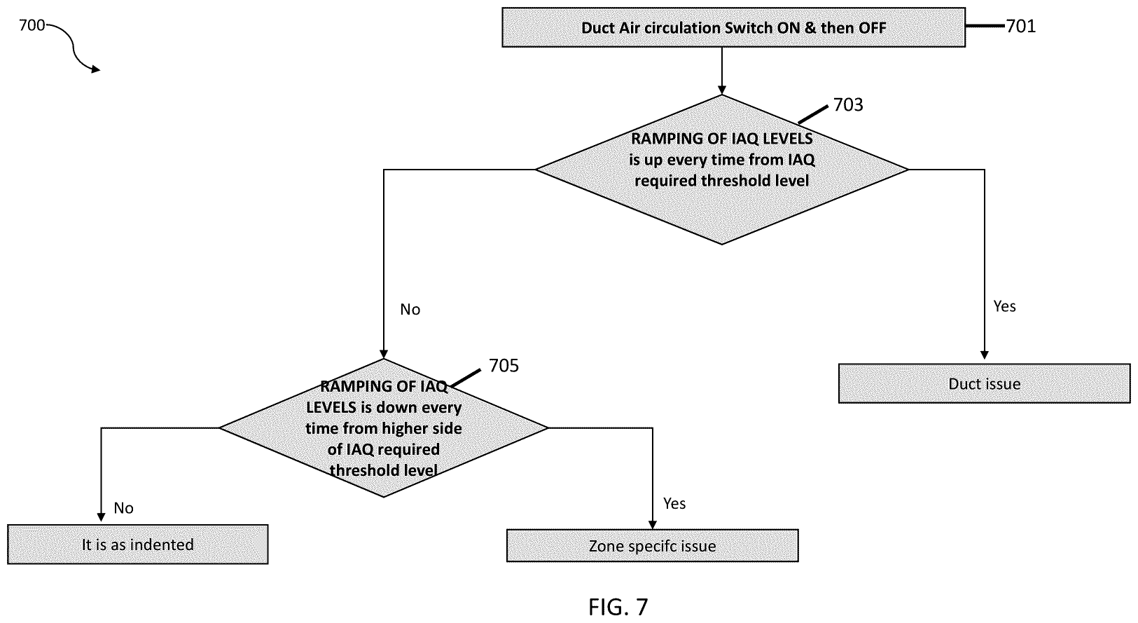

A stepwise explanation for the detection of one of the zone specific issues, the issue related to the at least one contaminated duct, or an intended issue using the ramping pattern determination process is provided below herein with the help of FIG. 7. FIG. 7 illustrates a flowchart of method steps for detecting at least one contaminated duct issue or zone specific issue using the ramping pattern.

At step 701 of the method 700, the processing unit 105 periodically controls the AHU 101 to turn on and turn off the duct air circulation to the one or more zones and checks for the IAQ PM2.5 pattern. The flow of the method 700 now proceeds to (step 703).

At step 703 of the method 700, the processing unit 105 checks whether the ramping of IAQ levels is up every time from the predefined IAQ required threshold level. In case a result of the determination at step 703 is NO, the flow of the method 700 now proceeds to (step 705). However, in case a result of the determination at step 703 is Yes, the processing unit 105 detects the at least one contaminated duct issue.

At step 705 of the method 700, the processing unit 105 checks whether the ramping of IAQ levels is down every time from the higher side of the predefined IAQ required threshold level. In case the result of the determination at step 705 is NO, then the processing unit 105 detects it as an intended issue. However, in case the result of the determination at step 705 is Yes, the processing unit 105 detects it as a zone specific issue.

The method 400 and 700 performed by the HVAC system 100 and its components helps in the easy and automatic identification of the location of dirty ducts within the indoor environment by having the HVAC system 100, and periodically notifying messages related to the contamination of the one or more ducts to the users as well as service team responsible for the maintenance of the HVAC system 100 via mobile applications and thermostat applications. Such notification message enables the users and the service team to take informed decisions for the maintenance of the HVAC system 100. The users and the service team may get benefitted from taking better decision upfront which may result in time saving and resources.

In addition, the method 400 and 700 performed by the HVAC system 100 and its components also provides a second level of verification to identify or classify either duct or a zone within the indoor environment that is causing IAQ deterioration or degradation.

As would be apparent to a person in the art, various working modifications may be made to the method in order to implement the inventive concept as taught herein.

Moreover, the actions of any flow diagram need not be implemented in the order shown; nor do all of the acts necessarily need to be performed. Also, those acts that are not dependent on other acts may be performed in parallel with the other acts.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

What is claimed is:

1. An HVAC system for detecting at least one contaminated duct among one or more ducts within an indoor environment, comprising:
an air handling unit (AHU);
a plurality of indoor air quality (IAQ sensors installed in the one or more Zones; and
a control device that includes a processor, a communication interface, and a display screen, wherein the processor is configured to:
control the AHU to turn-off air circulation to one or more zones within the indoor environment for a first time period;
control the plurality of IAQ sensors to measure a first set of IAQ values in the one or more zones when the air circulation to the one or more zones is turned off;
control the AHU to turn-on the air circulation to the one or more zones for a second time period;
control the plurality of IAQ sensors to measure a second set of IAQ values in the one or more zones when the air circulation to the one or more zones is turned on;
compare the first set of measured IAQ values with the second set of measured IAQ values;
detect the at least one contaminated duct among the one or more ducts based on a result of the comparison between the first set of measured IAQ values and the second set of measured IAQ values; and
notify, based on the detection of the at least one contaminated duct, a message indicating the at least one contaminated duct to a user on the display screen or a GUI of a mobile application using the communication interface.

2. The system as claimed in claim 1, wherein the processor is further configured to:
detect a specific location of the at least one contaminated duct within the indoor environment; and
notify, on the GUI of the mobile application using the communication interface or on the display screen, a message indicating the detected location of the at least one contaminated duct to the user.

3. The system as claimed in claim 1, wherein, for detecting the specific location of the at least one contaminated duct, the processor is further configured to:
re-control the AHU to turn-off the air circulation to corresponding zones among the one or more zones for a third time period;
control each of the plurality of IAQ sensors to measure a first set of individual IAQ values of the corresponding zones when the air circulation to the corresponding zones is turned off for the third time period;
re-control the AHU to turn-on the air circulation to the corresponding zones for a fourth time period;
control each of the plurality of IAQ sensors to measure a second set of individual IAQ values of the corresponding zones when the air circulation to the corresponding zones is turned-on for the fourth time period;
compare the first set of individual IAQ values with the second set of individual IAQ values;
detect the specific location of the at least one contaminated duct within the indoor environment based on a result of the comparison between the first set of individual IAQ values and the second set of individual IAQ values.

4. The system as claimed in claim 1, wherein the processor is further configured to:
determine, based on the comparison between the first set of individual IAQ values and the second set of individual IAQ values, at least one IAQ value among the second set of individual IAQ values that is greater than at least one IAQ value among the first set of individual IAQ values; and
detect the specific location of the at least one contaminated duct based on the at least one IAQ value that is determined among the second set of individual IAQ values.

5. The system as claimed in claim 1, wherein the processor is further configured to:
periodically send each of the first set of measured IAQ values, the second set of measured IAQ values, the first set of individual IAQ values, and the second set of individual IAQ values to a cloud server using the communication interface.

6. The system as claimed in claim 1, wherein the plurality of sensors includes at least one of a carbon dioxide sensor, PM2.5 sensor, PM10 sensor, or a total volatile organic compound (TVOC) sensor.

7. The system as claimed in claim 1, wherein the processor is further configured to:
determine a ramping pattern of the second set of measured IAQ values with respect to a predefined required IAQ level; and
detect one of a zone specific issue or an issue related to the at least one contaminated duct based on the determined ramping pattern.

8. A method for detecting at least one contaminated duct among one or more ducts within an indoor environment, comprising:
controlling an air handling unit (AHU) of an HVAC system to turn-off air circulation to one or more zones within the indoor environment for a first time period;
controlling a plurality of indoor air quality (IAQ) sensors of the HVAC system to measure a first set of IAQ values in the one or more zones when the air circulation to the plurality of zones is turned off;
controlling the plurality of IAQ sensors to measure a first set of IAQ values in the one or more zones when the air circulation to the one or more zones is turned off;
controlling the AHU to turn-on the air circulation to the one or more zones for a second time period;
controlling the plurality of IAQ sensors to measure a second set of IAQ values in the one or more zones when the air circulation to the one or more zones is turned on;
comparing the first set of measured IAQ values with the second set of measured IAQ values;
detecting the at least one contaminated duct among the one or more ducts based on a result of the comparison between the first set of measured IAQ values and the second set of measured IAQ values; and
notifying, based on the detection of the at least one contaminated duct, a message indicating the at least one contaminated duct to a user on the display screen or a GUI of a mobile application using the communication interface.

9. The method as claimed in claim 8, further comprising:
detecting a specific location of the at least one contaminated duct within the indoor environment; and
notifying, on the GUI of the mobile application using the communication interface or on the display screen, a message indicating the detected location of the at least one contaminated duct to the user.

10. The method as claimed in claim 8, wherein, for detecting the specific location of the at least one contaminated duct, the method further comprises:
re-controlling the AHU to turn-off the air circulation to corresponding zones among the one or more zones for a third time period;
controlling each of the plurality of IAQ sensors to measure a first set of individual IAQ values of the corresponding zones when the air circulation to the corresponding zones is turned-off for the third time period;
re-controlling the AHU to turn-on the air circulation to the corresponding zones for a fourth time period;
control each of the plurality of IAQ sensors to measure a second set of individual IAQ values of the corresponding zones when the air circulation to the corresponding zones is turned-on for the fourth time period;
comparing the first set of individual IAQ values with the second set of individual IAQ values;
detecting the specific location of the at least one contaminated duct within the indoor environment based on a result of the comparison between the first set of individual IAQ values and the second set of individual IAQ values.

11. The method as claimed in claim 8, further comprising:
determining, based on the comparison between the first set of individual IAQ values and the second set of individual IAQ values, at least one IAQ value among the second set of individual IAQ values that is greater than at least one IAQ value among the first set of individual IAQ values; and
detecting the specific location of the at least one contaminated duct based on the at least one IAQ value that is determined among the second set of individual IAQ values.

12. The method as claimed in claim 8, further comprising:
periodically sending each of the first set of measured IAQ values, the second set of measured IAQ values, the first set of individual IAQ values, and the second set of individual IAQ values to a cloud server using the communication interface.

13. The method as claimed in claim 8, wherein the plurality of sensors includes at least one of a carbon dioxide sensor, PM2.5 sensor, PM10 sensor, or a total volatile organic compound (TVOC) sensor.

14. The method as claimed in claim 8, further comprising:
determining a ramping pattern of the second set of measured IAQ values with respect to a predefined required IAQ level; and
detecting one of a zone specific issue or an issue related to the at least one contaminated duct based on the determined ramping pattern.
